(19) United States
(12) Patent Application Publication
(10) Pub. No.: US 2009/0118538 A1
(43) Pub. Date: May 7, 2009

(54) PROCESS FOR PREPARATION OF PURE ACITRETIN

(76) Inventors: Roberta PIZZOCARO, Garbagnate Milanese (IT); Cesare PELLEGATTA, Garbagnate Milanese (IT)

Correspondence Address: Steinfl & Bruno, 301 N Lake Ave Ste 810, Pasadena, CA, 91101, US

(21) Appl. No.: 12/266,374

(22) Filed: Nov. 6, 2008

(30) Foreign Application Priority Data
Nov. 7, 2007 (IT) MI2007A2128

(51) Int. Cl. C07C 51/42

(52) U.S. Cl. 562/494

(57) ABSTRACT

Processes and systems for the preparation of substantially pure acitretin based on reacting crude acitretin and an amine in presence of a suitable solvent to provide a salt that is then reacted with an organic acid to obtain substantially pure acitretin.

### CROSS-REFERENCE

This application claims priority to Italian Patent Application M12007A2128 filed on Nov. 7, 2007, the contents of which are incorporated herein by reference in their entirety.

### FIELD

The present disclosure relates to a process for the purification of acitretin

### BACKGROUND

Acitretin is a retinoic acid aromatic derivative, also known as (2E, 4E, 6E,8E)-9-(4-Methoxy-2,3,6-trimethylphenyl)-3,7-dimethyl-2,4,6,8-nonatetraenoic acid. Acitretin is currently used in the treatment of psoriasis, and in particular severe psoriasis, in adults. More particularly, acitretin can be used as an active agent against psoriasis in a monotherapy and/or combination therapy, and/or as a disease stabilizer in after-treatment approaches.

### SUMMARY

Provided herein are methods and systems for purification of acitretin, that allow preparation of substantially pure acitretin according to a process that in several embodiments is functionally simple and/or easy to scale up.

According a first aspect, a process for preparing substantially pure acitretin is disclosed. The process comprises:

providing crude acitretin;

providing an amine of formula

R₁R₂R₃N (I)

wherein each of R₁, R₂ and R₃ is, independently selected from the group consisting of hydrogen, a C₁-C₁ alkyl group, a C₃-C₈ cycloalkyl group, a phenyl, morpholino and a pyridino group;

salifying the crude acitretin with the amine of formula (I) in presence of a suitable solvent, thus obtaining a salt; and

adding an organic acid to the salt.

According to a second aspect, a system for preparing substantially pure acitretin, is disclosed. The system comprises at least two of: crude acitretin, the amine of formula (I) wherein each of R₁, R₂ and R₃ is, independently selected from the group consisting of hydrogen, a C₁-C₁ alkyl group, a C₃-C₈ cycloalkyl group, a phenyl, morpholino and pyridino group; and an organic acid. In particular, in the system the crude acitretin, the amine of formula (I) and the organic acid can be reacted to provide a substantially pure acitretin according to the method herein disclosed.

The details of one or more embodiments of the disclosure are set forth in the detailed description and examples below. Other features, objects, and advantages will be apparent from the description and the examples, and from the claims.

### DETAILED DESCRIPTION

Methods and systems are herein provided that allow preparation of substantially pure acitretin.

According to the present disclosure, the definition “substantially pure Acitretin” means Acitretin comprising less than 2%, preferably less than 1%, most preferably less than 0.5%, of undesired chemical impurities, which can be determined, for example, by HPLC.

According to the present disclosure, the definition “chemical impurities” means, in particular, cis-stereoisomers and Acitretin esters, like (2Z, 4E, 6E, 8E)-9-(4-methoxy-2,3,6-trimethylphenyl)-3,7-dimethyl-2,4,6,8-nonatetracnoic acid and ethyl (2E, 4E, 6E, 8E)-9-(4-methoxy-2,3,6-trimethylphenyl)-3,7-dimethyl-2,4,6,8-nonatetracnoate, as from the monograph in European Pharmacopoeia, 4a Edition, 2002.

In some embodiments, the method comprises:

a) salifying crude Acitretin with an amine of formula

R₁R₂R₃N (I)

wherein each of R₁, R₂ and R₃ is, independently, hydrogen, a C₁-C₆ alkyl

group, a C₃-C₈ cycloalkyl group, a phenyl, morpholino or pyridino group;

in presence of suitable solvent, and, in particular, an alcohol or water-alcohol solvent, and

b) adding an organic acid to the salt obtained in step a).

According to the present disclosure, the definition “crude Acitretin” comprises Acitretin in any form; for example, it means Acitretin containing a mixture of various cis-trans-isomers as impurities. Crude Acitretin can be prepared, for example, following the teaching of Hoffman-La Roche Patent U.S. Pat. No. 4,105,681, Ranbaxy Labs. Ltd. Patent U.S. Pat. No. 7,129,365, or Glenmark Pharm. Ltd. international patent application WO 2007/017720 each incorporated by reference in its entirety.

According to the present disclosure, the definition “C₁-C₆ alkyl” comprises, for example, methyl, ethyl, n-propyl, isopropyl, n-butyl, isobutyl, n-pentyl and n-hexyl.

According to the present disclosure, the definition “C₃-C₈ cycloalkyl” comprises, for example, cyclopropyl, cyclobutyl, cyclopentyl and cyclohexyl.

Preferably, in an amine of formula (II), each R₁, R₂ and R₃ is independently hydrogen or a C₁-C₄ straight alkyl group; more preferably, said amine is selected from triethylamine, diethylamine, dipropylamine and diisopropylamine; most preferably from triethylamine and diethylamine.

In some embodiments, acitretin is a compound formula (II),

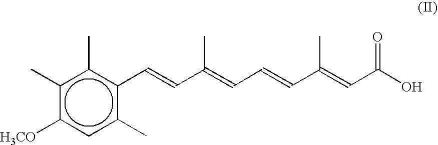

In some embodiments, the solvent used in step a) is preferably a water-alcohol solvent, and in particular a water alcohol solvent in which the alcohol is selected from methanol, ethanol, propanol, isopropanol or mixtures thereof; more preferably from ethanol and methanol.

In some embodiments, the reaction mixture of step a) can optionally be added with potassium hydroxide.

In some embodiments, the organic acid used in step b) is preferably an aliphatic carboxylic acid of formula R₄COOH, wherein R₄ is hydrogen or a C₁-C₄ straight alkyl group, preferably hydrogen or methyl, namely formic or acetic acid.

In some embodiments, step a) can be carried out adding crude Acitretin to a mixture of an alcohol or water-alcohol solvent and an amine of formula (I) as defined above, stirring until complete dissolution.

In some embodiments, step b) can be carried out adding the solution from step a) with an organic acid as defined above, until complete precipitation of substantially pure Acitretin, which is then recovered.

In some preferred embodiments, the present disclosure relates to a process for the preparation of substantially pure Acitretin, which comprises the following steps:

a) salifying crude acitretin with an amine of formula (I) as defined above wherein each of R₁, R₂ and R₃ is, independently, hydrogen or a C₁-C₆ alkyl group, in the presence of a water-alcohol solvent; and

b) adding an aliphatic carboxylic acid of formula R₄COOH, wherein R₄ is hydrogen or a C₁-C₄ straight alkyl group, to the salt obtained in the above step a).

In some most preferred embodiments, the present disclosure relates to a process for the preparation of substantially pure acitretin, which comprises the following steps:

a) salifying crude acitretin with an amine selected from triethylamine and diethylamine, in the presence of a water-alcohol solvent; and

b) adding an aliphatic carboxylic acid lected from acetic and formic acid to the salt obtained in step a).

The disclosure is illustrated in greater detail by the following examples. In particular, further advantages and characteristics of methods and systems herein disclosed will become more apparent from the following detailed disclosure of the Examples given by way or illustration only.

### EXAMPLES

### Example 1

A mixture of 260 ml of ethanol and 60 ml of water under nitrogen atmosphere is added with 38 g of triethylamine.

50 g of crude Acitretin is poured in the mixture with stirring, keeping stirring at temperature of 15° C.-25° C. until complete dissolution.

The solution is added with 220 ml of ethanol and slowly with 27 g of acetic acid, with cooling, thereby precipitating Acitretin.

The mixture is left under stirring for not less than 2 hours and the solid is filtered and washed with ethanol, to obtain Pure Acitretin (46 g on average).

### Example 2

A mixture of 400 ml of methanol and 60 ml of water under nitrogen atmosphere is added with 38 g of triethylamine.

50 g of crude Acitretin are poured in the mixture, stirring at a temperature of 15° C.-25° C. until complete dissolution.

The solution is slowly added with 27 g of acetic acid, with cooling, thereby precipitating Acitretin, which after about 2 hours is separated from reaction mixture by filtration to obtain about 45 g of pure Acitretin.

The examples set forth above are provided to give those of ordinary skill in the art a complete disclosure and description of how to make and use the embodiments of the methods and systems of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. In particular, all references cited in this disclosure are incorporated by reference to the same extent as if each reference had been incorporated by reference in its entirety individually.

It is to be understood that the disclosures are not limited to particular compositions or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing of the specific examples of appropriate materials and methods are described herein.

A number of embodiments of the disclosure have been described. Nevertheless, it will be understood that various modifications may be made without departing from the spirit and scope of the present disclosure. Accordingly, other embodiments are within the scope of the following claims.

1. A process for preparing substantially pure acitretin, the process comprising:
providing crude acitretin;
providing an amine of formula
R₁R₂R₃N (I)
wherein each of R₁, R₂ and R₃ is independently selected from the group consisting of hydrogen, a C₁-C₁ alkyl group, a C₃-C₈ cycloalkyl group, a phenyl, a morpholino, and a pyridino group;
salifying the crude acitretin with the amine of formula (I) in presence of a suitable solvent, thus obtaining a salt; and
adding an organic acid to the salt.

2. The process of claim 1, wherein each of R₁, R₂ and R₃ is independently selected from the group consisting of hydrogen or a C₁-C₆ alkyl group, and the organic acid is an aliphatic carboxylic acid of formula R₄COOH, in which R₄ is hydrogen or a C₁-C₄ straight alkyl group.

3. The process of claim 2, wherein the amine of formula (I) is diethylamine or triethylamine and the aliphatic carboxylic acid of formula R₄COOH is acetic or formic acid.

4. The process of claim 1, wherein the suitable solvent is a water-alcohol solvent.

5. The process of claim 4, wherein the water-alcohol solvent is selected from the group consisting of methanol, ethanol, propanol and isopropanol.

6. The process of claim 1, wherein salifying the crude acitretin with the amine of formula (I) is performed in presence of potassium hydroxide.

7. A system for preparing substantially pure acitretin, the system comprising at least two of:
crude acitretin;
an amine of formula
R₁R₂R₃N (I)
wherein each of R₁, R₂ and R₃ is, independently selected from the group consisting of hydrogen, a C₁-C₁ alkyl group, a C₃-C₈ cycloalkyl group, a phenyl, a morpholino, and a pyridino group; and
an organic acid
wherein the crude acitretin, the amine of formula (I) and the organic acid can be reacted according to the process of claim 1, to provide a substantially pure acitretin.

8. The system of claim 7, the system comprising the crude acitretin, the amine of formula (I) and the organic acid.

9. The system of claim 7, wherein each of R₁, R₂ and R₃ is, independently selected from the group consisting of hydrogen or a C₁-C₆ alkyl group, and the organic acid is an aliphatic carboxylic acid of formula R₄COOH, in which R₄ is hydrogen or a C₁-C₄ straight alkyl group.

10. The system of claim 7, wherein the amine of formula (I) is diethylamine or triethylamine and the aliphatic carboxylic acid of formula R₄COOH is acetic or formic acid.

11. The system of claim 7, wherein the suitable solvent is a water-alcohol solvent.

12. The system of claim 11, wherein the water-alcohol solvent is selected from the group consisting of methanol, ethanol, propanol and isopropanol.

13. The system of claim 7, the system further comprising potassium hydroxide.
